(19) United States
(12) Patent Application Publication
(10) Pub. No.: US 2023/0225887 A1
(43) Pub. Date: Jul. 20, 2023

(54) Unimodular Fenestrated Venous Stents

(71) Applicant: Arjun Jayaraj, Flowood, MS (US)

(72) Inventor: Arjun Jayaraj, Flowood, MS (US)

(21) Appl. No.: 17/492,665

(22) Filed: Oct. 3, 2021

(51) Int. Cl. A61F 2/856

(52) U.S. Cl. CPC A61F 2/856; A61F 2250/006

(57) ABSTRACT

Chronic venous insufficiency is an advanced form of venous disease affecting more than 2.5 million patients in the United States of whom approximately 500,000 have venous leg ulcers. [1-3] A majority of these patients have an obstruction in the deep veins of the pelvis either alone or with venous valve reflux that leads to the development of chronic venous insufficiency. Such obstruction often extends for varying lengths above the groin, at times all the way up to the right atrial chamber of the heart. Pathology of such obstruction could be narrowing of the aforementioned veins or could be a complete blockage (occlusion). Such treatment involves use of stent to open up the obstruction going from an area of good venous blood inflow to an area of good venous blood outflow.

### DISCUSSION OF PRIOR ART

1) Venous stents that are currently available do not have the ability to straddle across the iliocaval confluence without obstructing blood flow from the opposite side. The only way to do so is to use a double barrel configuration with one barrel for each side. However, since blood flow is related to the fourth power of radius (Poiseuille equation) use of a double barrel configuration results in a decrease in blood flow. E.g. a single 24 mm stent can carry eight (×8) times the flow that can be carried in 2 ×12 mm stents. This is important since inability to carry adequate amount of blood out from the leg and pelvis leads to persistent venous hypertension and residual symptoms even after the obstruction has been stented. So, use of a 24 mm stent is preferable to use of two 12 mm stents. But use of a single barrel stent means that the opposite vein gets blocked off with consequences in the form of blood clots and venous hypertension on the opposite side. This is the limitation of the currently available venous stents.

### SUMMARY OF INVENTION

The claim being made is for 3 types of stents:
Unimodular Fenestrated Femoroiliocaval Stent
Unimodular Fenestrated Perirenal caval Stent
Unimodular Fenestrated Perihepatic caval Stent

4) Each of these stents is unique in its ability to allow for blood flow from the opposite iliac vein (Unimodular Fenestrated Femoroiliocaval Stent); from both renal veins (Unimodular Fenestrated Perirenal caval Stent) and from the hepatic veins (Unimodular Fenestrated Perihepatic caval Stent) respectively. These stent configurations are important since it is key to allow for venous outflow from the iliac veins, renal veins or hepatic veins to prevent pooling of blood in the leg/pelvis, kidneys and liver. This can be accomplished by creating adequate size orifices in the configurations of these stents.

5) For the unimodular fenestrated femoroiliocaval stent, the stent is made by weaving together multiple wires made of alloy metal. A side orifice that can be identified by radiopaque markers allows for contralateral outflow or contralateral stent deployment thereby reducing risk of development of contralateral symptoms and/or contralateral deep venous thrombosis (DVT). The orifice is angled to allow for outflow from the opposite iliofemoral segment. This orifice is constructed such that it can be rotated to fit either the left or the right sides when deployed from the right or left sides respectively. The length should be adequate to extend from the common femoral vein to the inferior vena cava with diameters of the limb and orifice adequate enough to relieve venous hypertension on the ipsilateral side and prevent venous hypertension/deep venous thrombosis on the opposite side.

6) With regards to the Unimodular Fenestrated Perirenal caval Stent, the stent is made by weaving together multiple wires made of alloy metal. The fenestrae on the two sides accommodate inflow from the right and left renal veins. These orifices are almost diametrically opposite one another to enable blood outflow from the right and left renal veins and are identified by radio opaque markers. Additionally, the diameter of the stent should be such that it prevents an iatrogenic stenosis of the inferior vena cava. Another point in the invention is that the length of the Unimodular Fenestrated Perirenal caval stent enables it to be deployed into the unimodular fenestrated femoroiliocaval stent and thus allowing for reconstruction of the venous segment all the way from the common femoral vein in the groin to above the renal veins a length that can up to 15 inches.

7) Unimodular Fenestrated Perihepatic caval Stent is made by weaving together multiple wires made of alloy metal. The orifice (fenestrum) which can be identified by radio opaque markers accommodates inflow from the right, middle and left hepatic veins. Here again this stent can fit into the Unimodular Fenestrated Perirenal caval stent thereby allowing stenting from the groin all the way to the heart which is required in some patients.

### 8) DETAILED DESCRIPTION OF DRAWINGS

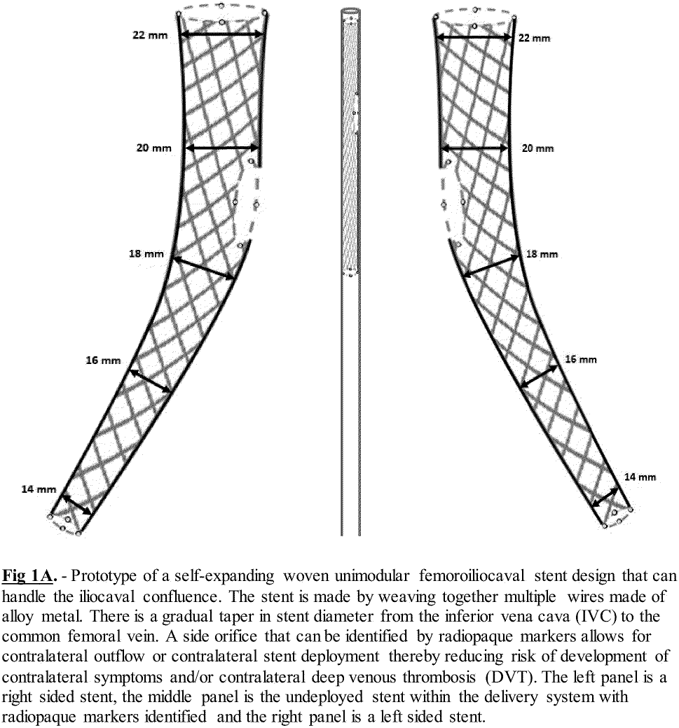

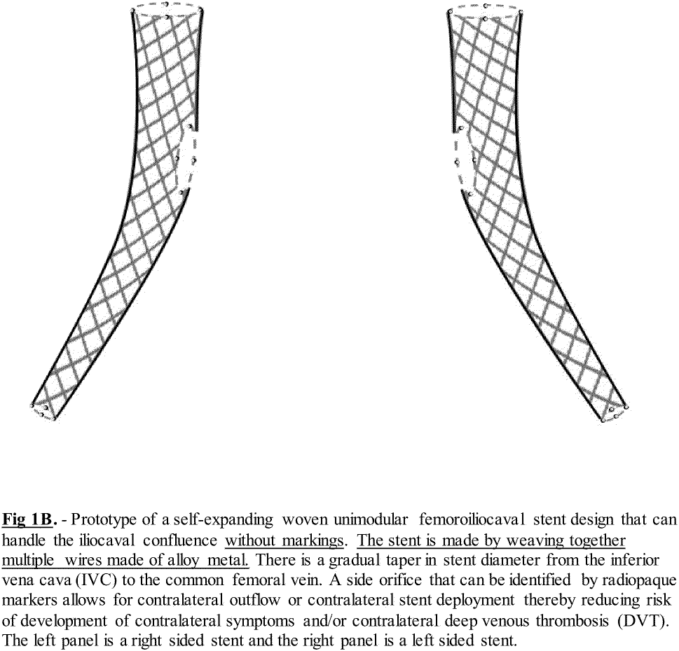

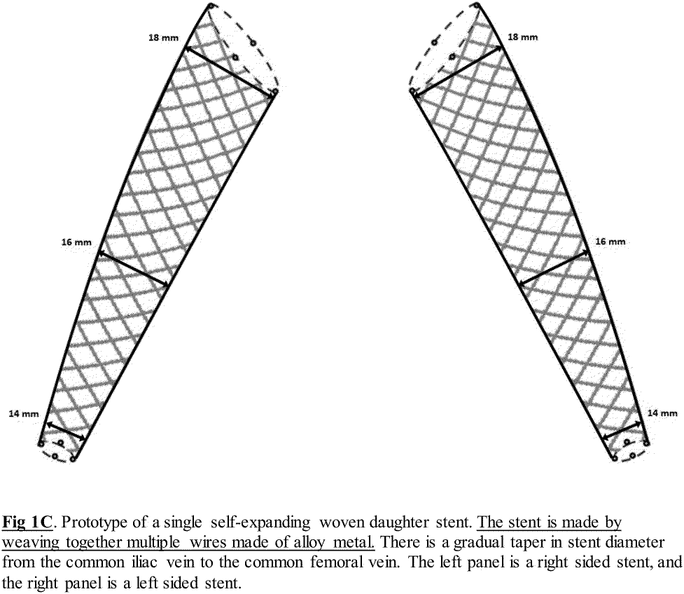

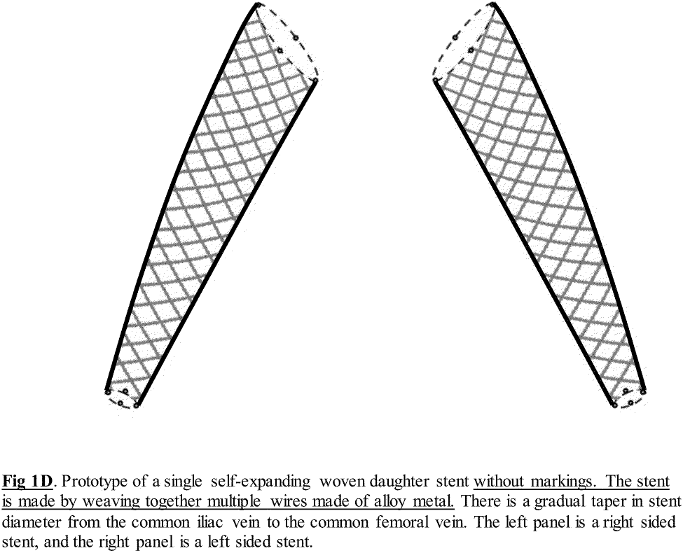

9) FIG. 1A. - Prototype of a self-expanding woven unimodular femoroiliocaval stent design that can handle the iliocaval confluence. The stent is made by weaving together multiple wires made of alloy metal. There is a gradual taper in stent diameter from the inferior vena cava (IVC) to the common femoral vein. A side orifice that can be identified by radiopaque markers allows for contralateral outflow or contralateral stent deployment thereby reducing risk of development of contralateral symptoms and/or contralateral deep venous thrombosis (DVT). The left panel is a right sided stent, the middle panel is the undeployed stent within the delivery system with radiopaque markers identified and the right panel is a left sided stent.

10) FIG. 1B. - Prototype of a self-expanding woven unimodular femoroiliocaval stent design that can handle the iliocaval confluence. The stent is made by weaving together multiple wires made of alloy metal. There is a gradual taper in stent diameter from the inferior vena cava (IVC) to the common femoral vein. A side orifice that can be identified by radiopaque markers allows for contralateral outflow or contralateral stent deployment thereby reducing risk of development of contralateral symptoms and/or contralateral deep venous thrombosis (DVT). The left panel is a right sided stent and the right panel is a left sided stent.

11) FIG. 1C. Prototype of a single self-expanding woven daughter stent. The stent is made by weaving together multiple wires made of alloy metal. There is a gradual taper in stent diameter from the common iliac vein to the common femoral vein. The left panel is a right sided stent, and the right panel is a left sided stent.

12) FIG. 1D. Prototype of a single self-expanding woven daughter stent without markings. The stent is made by weaving together multiple wires made of alloy metal. There is a gradual taper in stent diameter from the common iliac vein to the common femoral vein. The left panel is a right sided stent, and the right panel is a left sided stent.

13) FIG. 1E. Iliocaval stent configuration post deployment of a right sided self-expanding woven parent iliocaval stent and a left sided self-expanding woven daughter stent. Descriptions of both stent types are noted in FIG. 1A through FIG. 1D.

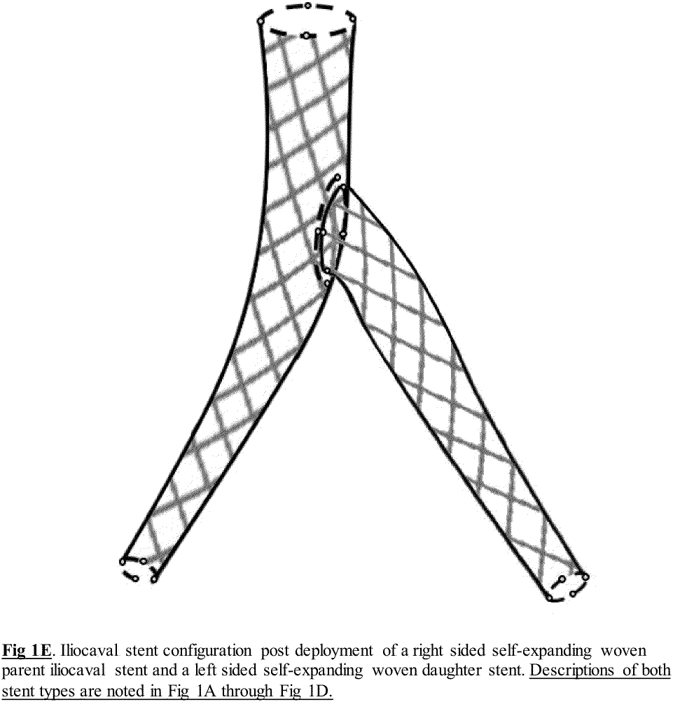

14) FIG. 2. Prototype of the self-expanding woven perirenal caval stent module. The stent is made by weaving together multiple wires made of alloy metal. The fenestrae on the two sides accommodate inflow from the right and left renal veins.

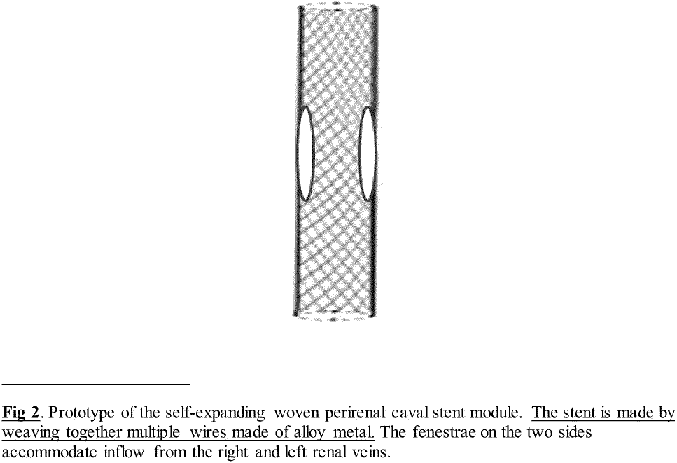

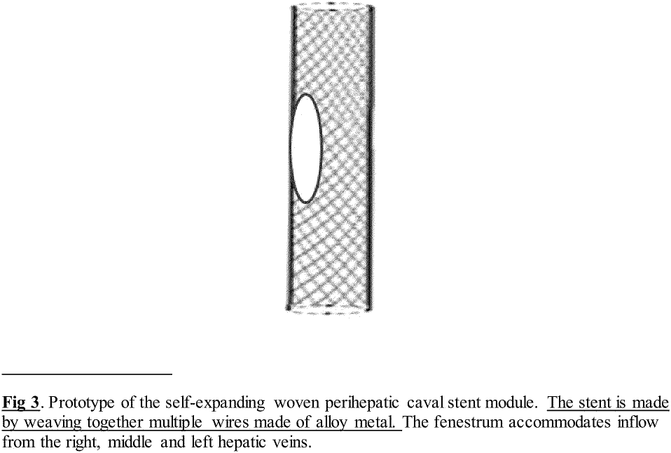

15) FIG. 3. Prototype of the self-expanding woven perihepatic caval stent module. The stent is made by weaving together multiple wires made of alloy metal. The fenestrum accommodates inflow from the right, middle and left hepatic veins.

16) Table 1. Stent configurations. Types I-II are femoro-ilio- caval configurations that allow for contralateral outflow/stenting. Lengths for femoro-ilio-caval stents allow for ~20 mm between the end of the stent and the original of the lowest renal vein. Stent extensions enable extending stent coverage in scenarios when the standard stent length comes short. Perirenal and perihepatic stent configurations are also depicted

1. What is claimed are unimodular fenestrated venous stents that extend from the common femoral vein (below groin) to the right atrium (heart). I claim a form of venous stent(s) that maintains normal anatomic venous blood flow following treatment of venous obstruction by use of fenestrae at the same time reducing the number of stents required (and thereby complications) by using a single module. The claims defining the invention are for a unique form of venous stent configuration(s). What is put forth is an alternative to conventional venous stents.

2. While pursuing stenting for pelvic and/or abdominal deep venous obstruction on one side it is important not to block the flow of blood from the opposite side. This is so because the veins that drain the right leg / pelvis and the left leg / pelvis have separate anatomic routes till they fuse to one another to form the inferior vena cava. Any extension of the stent into the inferior vena cava without providing an adequate channel for blood flow from the opposite side leads to obstruction of blood from the latter. Such obstruction can result in development of venous disease on the opposite side including but not limited to blood clots warranting interventions on an otherwise asymptomatic side. These contralateral complications which arise from j ailing of the contralateral blood outflow can be prevented by use of a unimodular stent that has a fenestrum to provide outflow from the opposite side. Such a stent is represented in FIGS. 1 A-C. Given that the vein increases in size as it goes up from the groin all the way up to the iliac confluence (approximately at the level of the umbilicus), such a unimodular fenestrated stent should allow a gradual cranial increase in stent diameter to accommodate for this. Additionally, given that at times stenting has to be extended all the way to the right atrium, two separate modules are available to go along with this limb module. The first is a perirenal module that has two fenestrae for allowing blood flow out of the renal veins into the inferior vena cava, and a second perihepatic module that has a single fenestrum to allow for outflow of blood from the liver. The perirenal caval stent module (FIG. 2) is constructed so that it can be deployed into the upper part of the limb module or as a standalone device. The perihepatic caval stent module (FIG. 3) is also constructed to be deployed into the upper part of the renal module or as a standalone device. Configuration for the different stent modules is considered in Table I. The stent configurations have been arrived at from careful measurements of computed tomographic scans dedicated to look at venous anatomy that patients underwent prior to venous stenting. Comparison of such CT measurements to the current gold standard for imaging of venous obstruction - intravascular ultrasound has been done before and demonstrated to be accurate. Together, these three modules enable coverage of diseased vein all the way from below the groin to the level of the right atrium and enables treatment of the entire complement of obstructive venous disease including pelvic and abdominal deep veins. Such a modular configuration for treatment of obstructive venous disease is currently not available.
